(19) United States
(12) Patent Application Publication
(10) Pub. No.: US 2022/0251499 A1
(43) Pub. Date: Aug. 11, 2022

(54) IMPROVED BUFFER FOR THE CAPACITATION OF SPERMATOZOA

(71) Applicants: Antonino PIPITO', Milano (IT); Elisabetta CASTAGNA, Padova (IT); Stefano TOZZO, Bolzano Vicentino (IT)

(72) Inventors: Antonino PIPITO', Milano (IT); Elisabetta CASTAGNA, Padova (IT); Stefano TOZZO, Bolzano Vicentino (IT)

(21) Appl. No.: 17/597,606

(22) PCT Filed: Jul. 28, 2020
(86) PCT No.: PCT/IB2020/057093

§ 371 Date: Jan. 13, 2022

(30) Foreign Application Priority Data
Jul. 29, 2019 (IT) 102019000013248

(51) Int. Cl. C12N 1/04; C12N 5/076; C07K 14/765

(52) U.S. Cl. CPC C12N 1/04; C12N 5/061; C12N 2500/60; C12N 2500/30; C07K 14/765

(57) ABSTRACT

A buffer for the capacitation of spermatozoa consisting of a buffer solution comprising the active ingredient Astaxanthin, in combination with serum albumin and a phosphate buffer solution made up of monobasic potassium phosphate KH₂PO₄ buffered with dibasic potassium phosphate K₂HPO₄.

### BACKGROUND OF THE INVENTION

The present invention relates to an improved buffer for the capacitation of spermatozoa.

The field of the invention is that of medically assisted fertilization techniques, in which the spermatozoa are collected in a culture medium, also called a buffer, consisting of a nourishing and protective fluid that must guarantee their viability and functionality. In particular, due to its optimizing characteristics, in said buffer the most motile spermatozoa can be selected (by means of the swimup technique), but, above all, they are made to “mature”, that is, to complete a series of modifications without which they would not be able to acquire their fertilizing capacity.

The problem found in commercial buffers such as, for example, the well-known Pure Sperm Wash buffer (PSW by Nidacon), already analyzed and described as one of the best on the market (Andrisani et al., Effects of various commercial buffers on sperm viability and capacitation—Systems Biology in Reproductive Medicine, 2014), is mainly that it does not allow the spermatozoon to reach an adequate level of capacitation for fertilization. In fact, even with this buffer, the sperm preparation process, which consists in restructuring the cell membrane and performing a specific enzymatic activation on the apical part (or head) of the spermatozoon, is not brought to a sufficient level to reach fertilization of the egg in a good percentage of cases.

A further drawback of the buffers available on the market is represented by their marked instability over time which means that, once the bottle is opened, the buffer must be used immediately and, in any case, in the shortest possible time.

The publications by Alessandra Andrisani et al. “Astaxanthin Improves Human Sperm Capacitation by Inducing Lyn Displacement and Activation”, Marine Drugs, vol. 13 no. 9, 25 Jan. 2015, pp. 5533-5551 (hereinafter referred to as A1) and Gabriella Donà et al. “Effect of Astaxanthin on Human Sperm Capacitation”, Marine Drugs, vol. 11, no. 6, 1 Jan. 2013, pp. 1909-1919 (hereinafter referred to as A2) describe studies on the mechanism of action of Astaxanthin on the capacitation of the human spermatozoon. These studies were carried out by adding Astaxanthin extemporaneously at the time of incubation to the medium containing the spermatozoa, without therefore any investigation into the temporal evolution of the biochemical parameters and motility related to denaturation of the product due to the instability of the final composition.

### SUMMARY OF THE INVENTION

The main object of the present invention is to provide a new buffer for the maturation of spermatozoa, which, unlike known buffers, offers a complete and balanced product containing all the substances capable of supporting spermatozoa in the maturation process.

Furthermore, another proposed object is that this new buffer is capable of determining an enhancement of the capacitation process, demonstrated by a higher percentage of spermatozoa more effective in the medically assisted fertilization techniques.

Finally, that this new buffer fully maintains its characteristics not only for the entire duration of an experiment but for a much longer time than the known formulas, with a shelf time of over 90 days in an intact bottle and more than 14 days after opening the bottle.

These objects are accomplished through the buffer of claim 1. Preferred embodiments of the invention will be apparent from the remaining claims.

The buffer of the invention offers the advantage of being ready for use without any need for extemporaneous additions, offers the advantage of an improved capacitation reaction, which allows a larger number of mature spermatozoa to be produced, with greater efficiency for medically assisted fertilization techniques; it presents a long stability in fully maintaining its functionality (more than 14 days after the bottle is opened); and, once prepared, it can be kept in a sealed bottle for more than 3 months.

In particular, this invention significantly enhances the sperm capacitation mechanism, especially when used with spermatozoa having abnormal ROS generation and, therefore, with a low probability of survival and maturation. In fact, according to the invention, the presence of Astaxanthin in combination with a phosphate buffer solution and serum albumin significantly restructures the membrane, effectively assisting the sperm capacitation process. Furthermore, the described combination of active ingredients gives stability to the buffer solution of the invention, since it considerably reduces the denaturation of the product, preventing natural oxidation of the ingredients and/or bacterial contamination.

The formulation of the ECB buffer, compared with known formulas, eliminates the need for extemporaneous preparation at the beginning of each single procedure. The formulation of the ECB buffer allows the Astaxanthin to be kept in a stable solution, thereby considerably simplifying the use of the buffer itself. In fact, the buffer contained in the packaging bottle can be used as it is by the operator, and all this represents a great advantage as regards the reliability and reproducibility of the results.

### DESCRIPTION OF THE PREFERRED EMBODIMENTS

These and other objects, advantages and characteristics result from the following formulation of the buffer of the invention, described in the following by way of a non-limiting example.

The buffer of the invention, called ECB (Enhancing Capacitation Buffer), is characterized by the presence of the active substance Astaxanthin, in combination with serum albumin, preferably of human origin, and a phosphate buffer solution consisting of monobasic potassium phosphate KH₂PO₄ buffered with dibasic potassium phosphate K₂HPO₄.

The buffer of the invention preferably includes 0.5 to 50 μM of Astaxanthin, 1 to 20 mg/ml of serum albumin, preferably human serum albumin, and 0.1 to 20 mM of KH₂PO₄—K₂HPO₄ solution, the latter in turn made up of variable concentrations of the compounds to determine a pH of between 7.00 and 9.00. In particular, said phosphate solution consists of 0.1 to 20 mM of KH₂PO₄—K₂HPO₄ buffer solution (pH range 7.00-9.00) and with an osmolarity of between 0.28 and 0.4 Osm.

Even more preferably the buffer of the invention includes 0.5 to 10 mM of MgSO₄, 5 to 50 mM of Ca-lactate, 0.1 to 20 mM of Na-pyruvate, 0.5 to 50 μM of Astaxanthin, 1 to 20 mg/ml of human serum albumin and 0.1 to 20 mM of KH₂PO₄—K₂HPO₄ solution.

Preferably the buffer solutions of the invention also comprise 5 to 50 mM of sodium bicarbonate NaHCO₃.

The Astaxanthin used in the buffer of the invention consists of a racemic mixture of synthetic Astaxanthin, having as general formula 3S,3S′-dihydroxy-β,β-carotene-4,4′-dione, (CAS 7542-45-2) preferably containing three stereoisomers (3R,3′R,3S,3′S and 3R,3′S) in a 1:1:2 ratio. It is, however, also possible to use natural, mono- or di-esterified Astaxanthin, naturally present in some algae or fungi with an antioxidant function, also with the general formula 3S,3S′-dihydroxy-β,β-carotene-4,4′-dione.

Below are some examples of buffer solutions of the invention, where:

μM represents micromoles/liter

mM represents millimoles/liter

Osm represents osmoles/liter

### EXAMPLE 1

| | | |
|---|---|---|
| Astaxanthin | 0.5-50 | μM |
| Phosphate buffer solution | 0.1-20 | mM |
| Human serum albumin | 1-20 | mg/ml |
| Saline solution pH 7.5 | up to 0.28 | Osm |

### EXAMPLE 2

| | | |
|---|---|---|
| Astaxanthin | 14 | μM |
| MgSO₄ | 1 | mM |
| Ca-lactate | 20 | mM |
| Na-pyruvate | 10 | mM |
| Phosphate buffer solution | 12 | mM |
| Human serum albumin | 12 | mg/ml |
| NaHCO₃ | 10 | mM |
| Saline solution pH 7.5 | up to 0.28 | Osm |

### EXAMPLE 3

| | | |
|---|---|---|
| Astaxanthin | 1 | μM |
| MgSO₄ | 4 | mM |
| Ca-lactate | 10 | mM |
| Na-pyruvate | 15 | mM |
| Phosphate buffer solution | 18 | mM |
| Human serum albumin | 5 | mg/ml |
| NaHCO₃ | 10 | mM |
| Saline solution pH 8.0 | up to 0.30 | Osm |

### EXAMPLE 4

| | | |
|---|---|---|
| Astaxanthin | 10 | μM |
| MgSO₄ | 9 | mM |
| Ca-lactate | 10 | mM |
| Na-pyruvate | 12 | mM |
| Phosphate buffer solution | 10 | mM |
| Human serum albumin | 9 | mg/ml |
| NaHCO₃ | 12 | mM |
| Saline solution pH 7.7 | up to 0.4 | Osm |

### EXAMPLE 5

| ECB buffer | | |
|---|---|---|
| Qualitative Composition | Concentrations | |
| MgSO₄ | 0.5-10 | mM |
| NaHCO₃ | 5-50 | mM |
| Ca-lactate | 5-50 | mM |
| Phosphate buffer solution | 0.1-20 | mM |
| (KH₂PO₄—K₂HPO₄) | | |
| Na-pyruvate | 0.1-20 | mM |
| Human Serum Albumin | 1-20 | mg/ml |
| Astaxanthin | 0.5-50 | μM |
| Saline solution | 0.28-0.4 | Osm |

| | |
|---|---|
| pH | 7.00-9.00 |

The effectiveness of the buffers in the capacitation of human sperm is measured in terms of: percentage of spermatozoa with slipping of the rafts on the sperm head (CTB) (Andrisani et al., Effects of various commercial buffers on sperm viability and capacitatiom—Systems Biology in Reproductive Medicine, 2014); percentage of cells with tyrosine phosphorylation on the head (p-Tyr); percentage of cells that obtain an acrosomal reaction (ARC); percentage of cells with progressive and non-progressive motility (M). These parameters represent the biochemical (CTB, p-Tyr, ARC) and mechanical-physical (M) characteristics of the spermatozoon, in turn characterized by the quantity of ROS free radicals that the spermatozoon produces in the buffer.

The parameters analyzed to describe the effectiveness of the various commercial buffers are those reported in the references as fundamental parameters of the capacitation process (Botto et al., Bicarbonates Induces Membrane Reorganization and CBR1 and TRPV1 Endocannabinoid Receptor Migration in Lipid Microdomains in Capacitating Boar Spermatozoa. The Journal of Membrane Biology, 2010; Donà et al., Endogenous reactive oxygen species content and modulation of tyrosine phosphorylation during sperm capacitation: ROS, Tyrphosphotylation and capacitation in human sperms. International Journal of Andrology 2011a; Nixon et al., Proteomic and functional analysis of human sperm detergent resistant membranes. Journal of Cellular Physiology 2011; Andrisani et al., 2014) and include:

the generation of ROS, which may aid membrane rearrangement by directly inducing lipid peroxidation, but which must never exceed a maximum value so as not to induce the degeneration of membranes and DNA; it is performed by detection with luminol (according to Donà et al., 2011a);

the slipping of the so-called lipid rafts, involved in the reorganization of the membrane architecture during capacitation; it is visualized by staining with subunit B of the cholera toxin (CTB) (according to indications given by Nixon et al., 2011; Andrisani et al., 2014);

the level of tyrosine phosphorylation (Tyr-P) of the proteins located in the sperm head, which must increase following the rearrangement of the aforementioned rafts as an indication of the correct functioning of proteins and enzymes located on the membrane (Donà et al., 2011b);

the percentage of cells that reach the acrosomal reaction (ARC), which represents the final step for the fusion of the sperm membrane with that of the oocyte (Donà et al., 2011a).

motility and kinetic parameters of spermatozoon, evaluated after 180 minutes of incubation in capacitating conditions in different buffers (M=percentage of spermatozoa with both progressive and non-progressive motility).

To better evaluate the efficacy of the buffer of the invention compared with the existing buffers represented by the PSW, the analytical technique of ROS production has allowed the semen samples to be classified in two groups, samples with a normal production of ROS (NG in the table below) and those with ROS generation problems (LPG in table 1 below), respectively:

TABLE 1

| | NG | LPG |
|---|---|---|
| ROS | 0.04-0.1 RLU | <0.04 RLU |
| production | | |

Table 1. Reference values for the classification of samples in the Normal Group (NG) or the Low Performing Group (LPG).

Endogenous production of ROS is detected by monitoring luminol luminescence during the incubation of spermatozoa under capacitating conditions. The values are expressed as the moving average of Relative Luminescence Units (RLU)/30s for 2×10⁶ spermatozoa.

Assessment

The samples used to perform tests according to the invention come from healthy donors (n=48) (age group, 28-45 years, average age 34.6) who have given their written informed consent, and met the criteria of normality according to the parameters described by the World Health Organization (World Health Organization, 2010). Once isolated and purified according to a density gradient (Donà et al., 2011a), the spermatozoa were divided into aliquots, washed separately with the different buffers and reanalyzed for concentration, motility, viability and morphology. The samples were then identified as belonging to the Normal Group (NG) or the Low Performing Group (LPG) on the basis of ROS production criteria as described previously (Donà et al., 2011a, 2011b). Aliquots of each sample were then processed in the different buffers for the parameters listed above (Donà et al., 2011b, 2011a; Andrisani et al., 2014).

Table 2 below lists the improvement percentages for each of the parameters evaluated, revealed by the ECB buffer of the invention with synthetic Astaxanthin, compared to the PSW buffer of the prior art, taken as a reference.

TABLE 2

| | | % increase | % increase | % increase | % increase | % increase |
|---|---|---|---|---|---|---|
| | | ECB vs | ECB vs | ECB vs | ECB vs | ECB |
| | | PSW | PSW | PSW | PSW | Old vs |
| | | fresh | 30 days | 60 days | 90 days | PSW |
| NG | CTB | 12 ± 3 | 11 ± 4 | 10 ± 3 | 10 ± 4 | 9 ± 3 |
| | p-Tyr | 9 ± 2 | 9 ± 4 | 8 ± 5 | 7 ± 3 | 7 ± 4 |
| | ARC | 19 ± 3 | 19 ± 4 | 17 ± 3 | 18 ± 4 | 15 ± 3 |
| LPG | CTB | 49 ± 4 | 48 ± 5 | 45 ± 4 | 43 ± 3 | 35 ± 4 |
| | p-Tyr | 41 ± 3 | 38 ± 4 | 40 ± 3 | 39 ± 5 | 33 ± 3 |
| | ARC | 52 ± 5 | 53 ± 3 | 50 ± 4 | 49 ± 4 | 47 ± 5 |

Table 2. Analysis of the biochemical parameters of the samples from volunteers assessed on the basis of the correct production of ROS, and defined as normal (NG, normal group) and from volunteers with abnormal ROS generation (LPG, low performing group).

The buffers used are the PSW (control buffer), and the ECB buffer used fresh, or 30, 60 or 90 days after preparation (ECB 30, 60, 90) or reused 2 weeks after opening the bottle (ECB Old). The parameters analyzed are: percentage of spermatozoa with slipping of rafts on the head (CTB), percentage of cells with tyrosine phosphorylation on the head (p-Tyr) and percentage of cells that obtain an acrosomal reaction (ARC). The data are expressed as a percentage increase of the values obtained with the ECB buffer under the various conditions, compared to the PSW buffer (taken as 100%). All values are expressed as mean±standard deviation.

The improvement, evident in the group of normal volunteers (NG), is expected in a range from 12 to about 19% with respect to that obtained with the PSW buffer, that proved to be an optimal buffer for the treatment of spermatozoa (Andrisani et al., 2014). In the LPG group treated with the ECB buffer of the invention, this improvement is 49% for the rearrangement of the membrane, and as much as 52% for the acrosomal reaction (ARC). Furthermore, even at 1, 2, 3 months after preparation, the ECB buffer shows that it guarantees the cells the same percentage increase of the various parameters, thus demonstrating that it maintains its optimal characteristics even after 3 months. The stability of the ECB buffer is further confirmed by evaluating the efficacy of a bottle used 14 days after opening, a condition that, on the contrary, caused a clear degradation of the PSW buffer, as demonstrated in previous studies. In fact, the data obtained demonstrate that the ECB buffer of the invention maintained the levels of the buffer used immediately after opening the bottle (Table 2).

When analyzed also for the percentage of motile cells (M), at the end of the incubation period (180′) the ECB buffer of the invention proved to be more effective than PSW, in inducing and maintaining progressive motility, increasing the percentage of motile cells, as shown in table 3 below.

TABLE 3

| | | % increase | % increase | % increase | % increase | % increase |
|---|---|---|---|---|---|---|
| | | ECB vs | ECB | ECB | ECB | ECB |
| | | PSW | 30 vs | 60 vs | 90 vs | Old vs |
| | | Fresh | PSW | PSW | PSW | PSW |
| NG | M | 3 ± 1 | 3 ± 2 | 3 ± 3 | 2 ± 2 | 2 ± 1 |
| LPG | M | 38 ± 3 | 36 ± 4 | 35 ± 3 | 33 ± 5 | 30 ± 4 |

Table 3. Analysis of spermatozoon motility of samples from volunteers assessed on the basis of correct ROS production, defined as normal (NG, normal group) and from volunteers with abnormal ROS generation (LPG, low performing group).

The buffers used are the PSW (control buffer), and the ECB buffer used fresh, or 30, 60 or 90 days after preparation (ECB 30, 60, 90) or reused 2 weeks after opening the bottle (ECB Old). The values represent the percentage of cells with progressive and non-progressive motility (M). The data are expressed as a percentage increase of the values obtained with the ECB buffer under the various conditions, compared to the PSW buffer (taken as 100%). All values are expressed as mean±deviation.

It may therefore be concluded that, on the basis of these results, the buffer of the invention has proved to be truly effective in preparing spermatozoa in that it significantly increases the percentage of capacitation of the samples. Considering also its effectiveness in preserving viability and increasing spermatozoon motility, the probability of successful fertilization within the scope of a medically assisted procreation treatment, or PMA, is undoubtedly enormously enhanced.

The preparation of the sperm in the washing buffers is a very important step in maintaining the correct physiological activities of the cells and, if the buffers used at the PMA centers for preparing the sperm are to induce optimal conditions to obtain the acrosomal reaction, the ECB buffer of the invention is far superior to the others, as can be seen from the comparison with the PSW buffer, already indicated as one of the most effective on the market.

In terms of the problem/solution criterion, the technical problem addressed is to increase sperm capacitation, by using a complete medium, perfectly balanced in its ingredients, which guarantee stability from preparation over time (over 90 days), and, even more importantly, 14 days after the bottle is opened.

This problem is not addressed, and therefore not solved, by any of the aforementioned documents A1-A2, which merely analyze the characteristics of a fresh buffer with a possible extemporaneous addition of freshly prepared Astaxanthin (see A1 p. 5538, Table 1; A2 p. 1916, par. 3.5 and 3.6).

Furthermore, from the comparison between A1 p. 5538, Table 1, PG Groups col. and Table 2, LPG col. of the description of the invention, it can be observed that the latter offers significant increases in p-Tyr and ARC, not only in the fresh buffer condition, but also 14 days after opening the bottle.

Therefore, considering A1 as the most relevant state of the art, none of the prior art documents teaches the combination of active ingredients that characterize the buffer of claim 1, with the aim of increasing sperm capacitation, under optimal conditions in a ready to use buffer, complete and stable for at least 90 days after preparation and up to at least 14 days after the bottle is opened.

In other words, it can be said that there are at least two essential elements that mark the difference between the known state of the art buffers and that of the invention:
stability, duration and reproducibility of the results;
use of Astaxanthin in a non-extemporaneous but structural way, unlike the known buffers.

The studies indicated in documents A1 and A2 do not provide any information on the temporal duration of the buffer used, as they only include the following steps:

suspension of spermatozoa in the chosen commercial buffer, for example PSW.

addition of Astaxanthin at time T₀, prepared extemporaneously.

recording of the data obtained from the experiment.

at the end of the experiment (T_{180′}), elimination of the extemporaneous solution prepared previously (including the spermatozoa) in the special laboratory waste.

This procedure confirms that the aim of both A1 and A2 was to study the behavior of Astaxanthin and its mechanism of action, while no attention was paid to the temporal evolution of the potential synergistic or antithetical action of Astaxanthin itself with the ingredients of the two commercial buffers used in A1 and A2.

It is therefore quite clear, both in A1 and in A2, that no information about the characteristics of the buffer known before time T₀ can be found, extrapolated or reconstructed.

For the same reason it is quite clear, both in A1 and in A2, that no information about the characteristics of the buffers known after the time T_{180′} can be found, extrapolated or reconstructed.

The total lack of information about the known buffers (for example, PSW+Astaxanthin) before and after extemporaneous preparation represents a basic and fundamental difference with respect to the buffer of the invention, and this lack of information does not provide the skilled in the art with the teachings that characterize the subject-matter of claim 1.

In addition, from the results of the comparison it can be concluded that the ECB buffer (subject-matter of the invention), compared to the extemporaneous buffers used in A1 and A2, has a shelf time of at least 90 days without any need for storage in the refrigerator; it does not require any dilution, the buffer is used as it is; no substance has to be added at the time of use, Astaxanthin is already present in the buffer; the product is absolutely stable and requires no controls or pH adjustments; the buffer maintains its characteristics for at least 2 weeks after the bottle is opened; and the replicability of the results of each bottle is guaranteed for at least 2 weeks after the bottle is opened.

1. Buffer for the capacitation of spermatozoa, characterized in that it consists of a buffer solution comprising the active ingredient Astaxanthin, in combination with serum albumin and a phosphate buffer solution consisting of monobasic potassium phosphate KH₂PO₄ buffered with dibasic potassium phosphate K₂H PO₄.

2. Buffer according to claim 1, characterized in that it comprises 0.5 to 50 μM of Astaxanthin, 1 to 20 mg/ml of human serum albumin and 0.1 to 20 mM of KH₂PO₄—K₂HPO₄ buffer solution.

3. Buffer according to claim 1, characterized in that said phosphate solution consists of 0.1 to 20 mM of KH₂PO₄ and K₂HPO₄ buffer solution (pH range 7.00-9.00) with an osmolarity of between 0.28 and 0.4 Osm.

4. Buffer according to claim 1, characterized in that it contains synthetic Astaxanthin with the formula 3,3′-dihydroxy-β,β-carotene-4,4′-dione, containing three stereoisomers (3R,3′R,3S,3′S and 3R,3′S).

5. Buffer according to claim 1, characterized in that said stereoisomers are present in a 1:1:2 ratio.

6. Buffer according to claim 1, characterized in that it contains natural, mono- or di-esterified Astaxanthin, with the formula 3,3′-dihydroxy-β,β-carotene-4,4′-dione.

7. Buffer according to claim 1, characterized in that it further comprises 5 to 50 mM of sodium bicarbonate NaHCO₃.

8. Buffer according to claim 1, characterized in that it comprises 14 μM Astaxanthin, 1 mM MgSO₄, 20 mM Ca-lactate, 10 mM Na-pyruvate, 12 mM phosphate buffer solution, 12 mg/ml human serum albumin, 10 mM NaHCO₃ and up to 0.28 Osm saline solution pH 7.5.
